(19) United States
(12) Patent Application Publication
(10) Pub. No.: US 2008/0214816 A1
(43) Pub. Date: Sep. 4, 2008

(54) Process for Preparing Ziprasidone

(76) Inventors: Carme Burgarolas Montero, Barcelona (ES); Jordi Puig Serrano, Barcelona (ES); Carme Arnalot Aguilar, Barcelona (ES); Jordi Bosch Illado, Barcelona (ES)

Correspondence Address: LADAS & PARRY LLP, 26 WEST 61ST STREET, NEW YORK, NY, 10023, US

(21) Appl. No.: 11/663,481

(22) PCT Filed: Sep. 15, 2005
(86) PCT No.: PCT/EP05/54588

§ 371 Date: Nov. 20, 2007

(30) Foreign Application Priority Data
Sep. 29, 2004 (ES) 200402315

(51) Int. Cl. C07D 417/14; C07C 255/03

(52) U.S. Cl. 544/368; 558/435

(57) ABSTRACT

Process for preparing ziprasidone. The present invention concerns a process for the preparation of 5-(2-(4-(1,2-benzisothiazol-3-yl)-1-piperazinyl)ethyl)-6-chloro-1,3-dihydro-2H-indol-2-one of the formula I, or a pharmaceutically acceptable acid addition salt, solvate, hydrates or clathrate thereof, said process comprising reacting a compound of formula II wherein X is a halogen atom, with a compound of formula III, said compound of formula III being the free base or an addition salt with an organic or inorganic acid, wherein said process is characterized in that said compounds according to formulas II and III are reacted in the presence of a neutralizing agent, and are reacted in a solvent comprising acetonitrile.

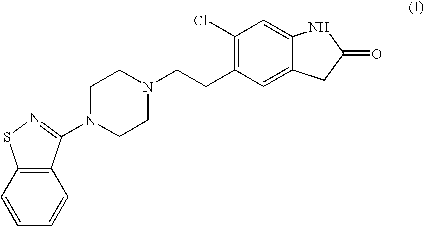

### FIELD OF THE INVENTION

The invention belongs to the pharmaceutical field, in particular to a new process for obtaining ziprasidone drug.

### BACKGROUND OF THE INVENTION

The present invention relates to a process for preparing ziprasidone or pharmaceutically acceptable acid addition salts, hydrates, solvates or clathrates thereof. Ziprasidone is the common name for 5-[2-[4-(1,2-benzisothiazol-3-yl)-1-piperazinyl]ethyl]-6-chloro-1,3-dihydro-2H-indol-2-one, of formula (I):

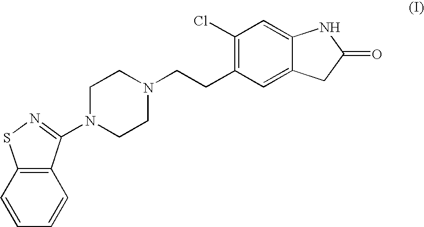

Ziprasidone is an active pharmaceutical ingredient having neuroleptic activity.

EP281309-A1 refers to a group of compounds including ziprasidone and salts thereof and to a process to prepare them by means of a coupling reaction performed in organic polar solvents like ethanol, N,N-dimethylformamide (DMF) and methylisobutylketone (MIBK). In Example 16 of EP281309-A1 a process for preparing ziprasidone hydrochloride in hemihydrated form is disclosed, which comprises a coupling reaction as shown in the following scheme:

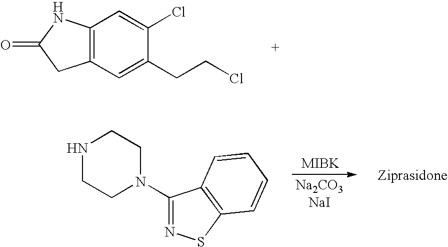

The reaction is carried out in methylisobutylketone as the solvent, in the presence of an excess of sodium carbonate as neutralizing agent, and sodium iodide as a catalyst. The yield of ziprasidone hydrochloride in hemihydrate form according to Example 16 of EP281309-A1 is 20%, very low for industrial implementation. This low yield indicates the presence of high amounts of by-products, which makes costly purification procedures like chromatographic techniques to isolate ziprasidone necessary.

EP584903-A1 refers to a different approach to prepare ziprasidone, in which the coupling reaction is carried out in water in the presence of an excess of sodium carbonate as neutralizing agent.

According to the prior art, the manufacture of ziprasidone in organic polar solvents is not satisfactory for industrial implementation because the yields are very low and the presence of high amounts of undesired by-products makes the use of non-economical purification procedures to isolate the product in the required quality specifications necessary.

Thus, it was the objective of the present invention to provide an alternative process for the production of ziprasidone or pharmaceutically acceptable acid addition salts, hydrates, solvates or clathrates thereof, characterized by a high yield, and reduced formation of by-products, to avoid special purification procedures like column chromatography.

### BRIEF DESCRIPTION OF THE INVENTION

The inventors of the present invention have surprisingly found that when an acetonitrile comprising solvent is selected as the polar organic solvent for the production of ziprasidone, it is possible to obtain ziprasidone in high purity. This is in contrast to all other organic solvents previously used, which resulted in a poor evolution of the reaction (i.e. poor yields) and/or complex mixtures of reaction products. In other words, the crude product obtained using the prior art teaching is far more impure than the crude product obtained using the process according to the present invention. Thus, surprisingly the present invention provides ziprasidone at high yield, i.e. less diluted and at the same time reduces the formation of by-products. Therefore, the present invention avoids the need for special purification procedures like column chromatography. Furthermore, in using acetonitrile as a solvent it has been surprisingly found that the reaction product does not degrade even when subjected to high temperature in a polar solvent.

Thus, the new process is clearly advantageous over the previously described process based on using organic polar solvents and provides a useful industrial alternative to the process of the prior art based on using water as the solvent.

The present invention relates to a process for the preparation of ziprasidone, i.e. 5-[2-[4-(1,2-benzisothiazol-3-yl)-1-piperazinyl]ethyl]-6-chloro-1,3-dihydro-2H-indol-2-one of the formula I

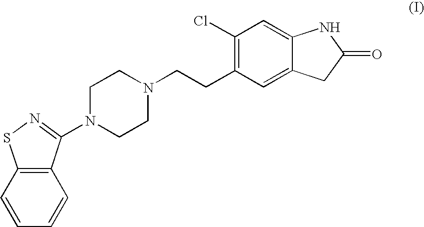

or a pharmaceutically acceptable acid addition salt, solvate, hydrate or clathrate thereof, said process comprising reacting a compound of formula II

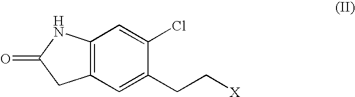

wherein X is a halogen atom, preferably chlorine, with a compound of formula III

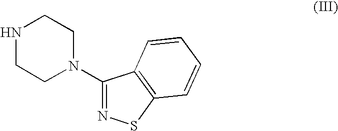

said compound of formula III being the free base or an addition salt with an organic or inorganic acid, wherein said process is characterized in that said compounds according to formulas II and III are reacted in the presence of a neutralizing agent, preferably selected from alkali or alkaline earth metal carbonates, bicarbonates, and tertiary amines, more preferably selected from sodium carbonate or N,N-diisopropylethylamine, in a solvent comprising acetonitrile. Preferably the solvent is acetonitrile.

The ziprasidone of formula I so obtained can be transformed if desired, by conventional means, in a pharmaceutically acceptable acid addition salt, solvate, hydrate or clathrate thereof.

It is preferred that compound (III) is used as its addition salt with an acid selected from hydrochloric acid, hydrofluoric acid, hydrobromic acid, hydroiodic acid, methanesulfonic acid, trifluoromethanesulfonic acid and/or trifluoroacetic acid, preferably hydrochloric acid.

In one embodiment, the compound according to formula III is used as the free base, and the neutralizing agent is used in an amount of one to four molar equivalents based on the compound according to formula III.

In an alternative embodiment the compound according to formula III is used as an addition salt with an organic or inorganic acid, and the neutralizing agent is used in an amount of two to four molar equivalents based on the compound according to formula III.

The reaction of the present invention can be carried out in the presence of sodium iodide as a catalyst. The piperazine derivative according to formula (III), the alkyl halide according to formula (II), the neutralizing agent and NaI are mixed in an acetonitrile comprising solvent, and preferably a reaction temperature is selected from 80° C. to 180° C. Preferably, the reaction is kept at the selected temperature for 3 to 80 h, more preferably for 5 to 30 h. In one embodiment the reaction process is performed in a sealed reactor.

Subsequently to keeping the reaction at the selected temperature, the reaction mixture is cooled, filtered and the solid is washed with acetonitrile. Alternatively, or in combination, the free base according to formula I is treated with any one, or subsequently with both of boiling water and boiling tetrahydrofuran.

In a further embodiment the reaction is carried out in the presence of sodium iodide as a catalyst in an amount close to stoichiometric amount, preferably at or close to the acetonitrile reflux temperature and further preferably at or close to atmospheric pressure.

The present invention also relates to the use of acetonitrile as a solvent in a process to produce ziprasidone.

### DETAILED DESCRIPTION OF THE INVENTION

Surprisingly, in spite of the disclosure of EP281309-A1, the selection of acetonitrile as the polar organic solvent allowed the preparation of ziprasidone in unexpected high yields, close to 60%, and high purity, suitable to be used in pharmaceutical formulations with only conventional minor purification treatments.

Therefore, it is essential for the process of the present invention that the reaction for preparing ziprasidone is carried out in solvent comprising acetonitrile, preferably in acetonitrile as the single solvent. According to the present invention, a solvent comprising acetonitrile is a solvent comprising at least 25%, more preferably more than 50%, more preferably more than 75%, more preferably more than 90% and most preferably 100% of acetonitrile.

The starting compounds of formula (II) and (III) can be prepared following the methods described in EP281309-A1.

For the purposes of the present invention compounds (II) and (III) can be present in equal molar amounts or, alternatively, one of them can be present in an excess. Said excess can be in the range of 0 to 3 molar equivalents, preferably from 0 to 1 molar equivalents. An excess of 0 molar equivalents corresponds to equal molar amounts.

A neutralizing agent is used to neutralize the hydrohalic acid, which is formed in the coupling reaction. The neutralizing agent is an organic or inorganic base, preferably selected from the group comprising alkali or alkaline earth metal carbonates, such as sodium carbonate or potassium carbonate; bicarbonates such as sodium bicarbonate; and/or tertiary amines such as triethylamine or diisopropylethylamine. Combinations of neutralizing agents can be used.

In a more preferred embodiment of the process of the invention, the neutralizing agent is sodium carbonate or diisopropylethylamine.

Preferably the neutralizing agent is used in excess. Most preferably the process of the invention involves the use of from two to four molar equivalents of a neutralizing agent based on the starting material. When the compound according to formula III is used as the free base, the neutralizing agent is used in an amount of at least 1 molar equivalent, preferably 1 to 4 molar equivalents based on the compound according to formula III. When the compound according to formula III is used as an addition salt with an organic or inorganic acid, the neutralizing agent is used in an amount of at least 2 molar equivalents, preferably 2 to 4 molar equivalents based on the compound according to formula III.

Preferably sodium iodide is used as a catalyst in the process of the invention.

In a further embodiment the reaction is carried out in the presence of sodium iodide as a catalyst in an amount close to stoichiometric amount, preferably at or close to the acetonitrile reflux temperature and further preferably at or close to atmospheric pressure; “close to” the reflux temperature of acetonitrile here is intended to cover a temperature which is 10° C. over or below the reflux temperature of acetonitrile.

Addition salts with an organic or inorganic acid of the compound of formula III according to the present invention comprise addition salts with an acid selected from hydrochloric acid, hydrofluoric acid, hydrobromic acid, hydroiodic acid, methanesulfonic acid, trifluoromethanesulfonic acid and/or trifluoroacetic acid, preferably hydrochloric acid.

The process of the invention is preferably carried out at a temperature from 80° C. to 180° C.

When the selected reaction temperature is in excess of the boiling point of the solvent, the reaction process can be performed in a sealed reactor. As an example, above the boiling point of acetonitrile (about 80° C.) it is necessary to carry out the reaction in a pressure vessel (i.e. a sealed reactor), wherein the pressure can increase from atmospheric pressure to about 1500 kPa. The pressure of the reaction in the sealed reactor will be determined by the selected reaction temperature and solvent. Pressures routinely achieved by a process according to the present invention are in the range of 100-1200 kPa, more often 200-1000 kPa.

The reaction mixture is heated for a time sufficient to allow the reaction to proceed, generally at least about 3 to 80 hours, preferably from 5 to 30 hours. Then the reaction mixture is cooled to room temperature and the crude product is filtered off. Subsequently the crude product can be washed with acetonitrile.

If desired an alternative or additional step can be included for removing eventual inorganic salts by treating the crude product with water at a temperature from 70° C. to boiling, preferably boiling.

Alternatively, or in combination, eventual residual starting materials can be also removed, if necessary, by treating the crude product with tetrahydrofuran at a temperature from 40° C. to boiling, preferably boiling.

According to the process of the invention, ziprasidone base is obtained in high yield, close to 60% (i.e. for example 50-70%, preferably more than 60%), regarding the starting materials, and in good enough quality for using it in pharmaceutical preparations. In this context, “good enough quality” means, that the ziprasidone is obtained in such purity, that no complicated additional purification steps, such as column chromatography, are required.

The pharmaceutically acceptable acid addition salts of ziprasidone or their hydrates, solvates or clathrates can be prepared in a conventional manner, for example by treating a solution or suspension of ziprasidone base with the acceptable acid. Examples of these pharmaceutically acceptable acid addition salts, solvates, hydrates or clathrates according to the present invention comprise ziprasidone mesylate, ziprasidone mesylate trihydrate, ziprasidone mesylate dihydrate, ziprasidone esylate, ziprasidone tartrate, ziprasidone besylate, ziprasidone tosylate, ziprasidone hydrochloride, ziprasidone hydrochloride hemihydrate, ziprasidone hydrochloride monohydrate, ziprasidone maleate, ziprasidone acetate.

The process of the invention allows the preparation of ziprasidone or pharmaceutically acceptable acid addition salts, solvates, hydrates or clathrates thereof in high yields, reducing the presence of by-products and providing ziprasidone in high purity suitable to be used in pharmaceutical preparations only with conventional minor purification treatments.

Ziprasidone free base is very insoluble in common solvents. This is demonstrated e.g. in example 2 of U.S. Pat. No. 5,338,846 and corresponding example 3 of EP 584 903, where it is disclosed that 1 kg of ziprasidone base requires 9 to 10 gallons of tetrahydrofurane (one US gallon corresponds to 36.2 litres; one UK gallon corresponds to 43.4 litres) and reflux temperature (66° C.) to obtain a solution of ziprasidone base. Such very large volumes of solvent and the filtration temperature near to reflux temperature are disadvantageous for industrial implementation. Thus, it would be advantageous to obtain a derivative of ziprasidone base that is more soluble than ziprasidone base.

To achieve this aim, the ziprasidone free base can be reacted with maleic acid or acetic acid, preferably in an amount of 1 0.5 to 3 molar equivalents, preferably 1 to 2 molar equivalents, and most preferably 1.1 to 1.6 molar equivalents to obtain an acid addition salt of the following formula (IV):

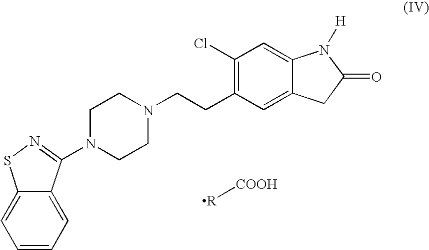

wherein R is

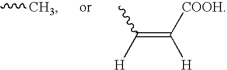

Then, the acid addition salt according to the above formula (IV) is separated from insoluble components of the composition, preferably by filtration.

Alternatively, or in addition, the acid addition salt according to the above formula (IV) can be further treated with a decolorizing agent, preferably at least one selected from alumina, activated alumina, silica and charcoal.

The acid addition salt according to the above formula (IV) can be reacted with an acid, preferably selected from hydrochloric acid, hydrobromic acid and methanesulphonic acid, most preferably hydrochloric acid, in order to obtain an acid addition salt according to the following formula (V):

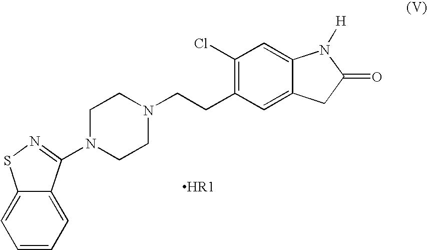

wherein R1 is halogen or CH₃SO₃.

The solution of the addition salt according to formula IV can be treated with hydrochloric acid or with hydrogen chloride or with a solution of hydrogen chloride in order to precipitate ziprasidone hydrochloride.

Alternatively, the solution of the addition salt according to formula IV can be treated with a base in order to precipitate ziprasidone base, which is then converted to the corresponding acid addition salt according to formula (V). Suitable bases comprise sodium hydroxide, potassium hydroxide, sodium carbonate, potassium carbonate sodium bicarbonate, potassium bicarbonate and ammonium hydroxide.

The acid addition salt according to the above formula (V) can be further purified by using at least one organic solvent, preferably selected from isopropanol, tetrahydrofuran, n-butanol and butan-2-one.

The following examples are provided to illustrate the invention.

### EXAMPLES

The following analytical chromatographic HPLC method is used to test the purity of ziprasidone:

The test is carried out in a Kromasil C8 column of 5 μm and 250×4.6 mm. The mobile phase is prepared by mixing 370 ml of acetonitrile with 630 ml of buffer at a pH=3.0, which is prepared from 1.2 g KH₂PO₄ and 0.7 g of 1-pentanesulfonic acid sodium salt dissolved in 630 ml of water, adjusting the pH with H₃PO₄. This mobile phase is mixed and filtered through a 0.22 μm nylon filter under vacuum.

The chromatograph is equipped with a UV detector set at 229 nm and the flow rate is 1.0 ml per minute at room temperature. The samples are prepared by dissolving the appropriate amount of sample to obtain 0.5 mg per ml of a mixture of acetonitrile/trifluoroacetic acid 19.6:0.4 v/v and 20 μl are injected.

### Example 1

### Preparation of Ziprasidone Base

88.7 g (0.837 mols, 3.21 molar equivalents) of sodium carbonate, 600 ml of acetonitrile and 66.7 g (0.261 mols, 1.0 molar equivalent) of 3-(1-piperazinyl)-1,2-benzisothiazole hydrochloride [hydrochloride of the compound of formula (III)] are added into a beaker equipped with a magnetic stirrer. The resulting white suspension is stirred for 10 minutes. At this point 60.0 g (0.261 mols, 1.0 molar equivalent) of 5-(2-chloroethyl)-6-chloro-1,3-dihydro-indole-2-(2H)-one [compound of formula (II) wherein X is chlorine] and 0.3 g (0.002 mols, 0.008 molar equivalents) of NaI are added. The resulting brown suspension is charged into a 1 L reactor vessel, which is purged with nitrogen and heated to 120-125° C. (internal pressure increases to 400-500 kPa) for 25 hours. The reaction is cooled to room temperature, stirred for 30 minutes, filtered and the solid washed with acetonitrile. A wet mixture of zipradisone and inorganic salts is obtained.

The resulting wet mixture is stirred with 675 ml of water at reflux temperature for 1 h to remove inorganic salts. The suspension is cooled at room temperature, stirred for 30 minutes and filtered. The solid is washed with water, and 140 g of wet solid (corresponding to 87 g of dry material) are obtained.

The wet solid is stirred again with water at reflux temperature for 1 h to remove residual inorganic salts. The suspension is cooled to room temperature, stirred for 30 minutes and filtered. The solid is washed with water, and 170 g of wet solid (corresponding to 81 g of dry material) are obtained. HPLC analysis reveals a purity of 97.8%.

To remove starting materials present in the wet solid obtained in the previous step, it is stirred twice with 400 ml of tetrahydrofuran at reflux temperature. The solution is cooled to room temperature, stirred for 30 minutes and filtered. The solid is washed twice with 40 ml of tetrahydrofuran at room temperature and 60 g of wet solid, corresponding to 54.8 g of dry material, are obtained.

The solid obtained is ziprasidone base having a purity of 99.4% by HPLC and the global yield from the starting compound (II) or (III) is 51% molar. Potentiometric titration with HClO₄: 100.03%

Optionally, Ziprasidone base could be converted to ziprasidone hydrochloride.

### Example 2

### Preparation of Ziprasidone Base

119 ml (90.1 g, 0.698 mols, 3.21 molar equivalents) of N,N-diisopropylethylamine, 500 ml of acetonitrile and 55.8 g (0.218 mols, 1.0 molar equivalents) of 3-(1-piperazinyl)-1,2-benzisothiazole hydrochloride (addition salt of compound of formula (III) and hydrochloric acid) are added into a beaker equipped with a magnetic stirrer. The resulting suspension is stirred for 10 minutes. At this point 50 g (0.217 mols, 1.0 molar equivalent) of 5-(2-chloroethyl)-6-chloro-1,3-dihydro-indole-2-(2H)-one (Compound of formula (II) wherein X is chlorine) and 0.26 g (1.174 mmols, 0.008 molar equivalents) of NaI are added. The resulting brown suspension is charged into a 1 L reactor vessel, which is heated to 121-122° C. (internal pressure increases to 200 kPa) for 25 hours. The reaction is cooled to room temperature and filtered. The solid is washed with acetonitrile, and 56 g of a wet solid are obtained.

The resulting wet solid is stirred with 4 volumes of water at reflux temperature for 1 h to remove inorganic salts. The suspension is cooled to room temperature and filtered. The solid is washed with water. Ziprasidone base is obtained in 56% molar yield and the purity is 97.8% by HPLC.

### Example 3

### Large Scale Preparation of Ziprasidone Base

Into a 1001 Hastelloy reactor are loaded:
8 kg (31.3 mols 1.0 molar equivalent) of 3-(1-piperazinyl)-1,2-benzisothiazole hydrochloride [hydrochloride of compound of formula (III)],
8.64 kg (37.5 mols, 1.2 molar equivalents) of 5-(2-chloroethyl)-6-chloro-1,3-dihydro-indole-2-(2H)-one [compound of formula (II) wherein X is chlorine],
10.6 kg (100 mols, 3.20 molar equivalents) of sodium carbonate,
0.038 kg (0.25 mols, 0.008 molar equivalents) of NaI

The reactor is closed and blanketed with vacuum/nitrogen. Then, 56.3 kg of acetonitrile are loaded and the mixture is stirred for 10 minutes. The reactor is heated to reflux (80-82° C.). Then the reactor is closed and continued to be heated up to 120-125° C. (internal pressure increases to 300 kPa). The reaction mixture is kept under these conditions for 25 hours. Then the content is cooled down to room temperature and the solid is centrifuged and washed with 2×12 kg of acetonitrile. A wet solid containing ziprasidone base and inorganic salts is obtained.

The resulting solid is loaded in a 1001 Hastelloy reactor. The reactor is blanketed and 52 kg of water are loaded. The suspension is stirred at reflux conditions (80-85° C.; due to the presence of acetonitrile) for 1 h to remove inorganic salts. The suspension is cooled down to room temperature, stirred for 30 minutes and the solid is centrifuged and washed with 2×9 kg of water. 17.97 kg of wet solid are obtained.

The wet solid from the previous step is loaded in a 1001 Hastelloy reactor. The reactor is blanketed and 57 kg of tetrahydrofuran are loaded. The suspension is stirred at reflux conditions for 1 h. The suspension is cooled down to room temperature, stirred for 30 minutes and the solid is filtered through a Nutsche Filter and washed with 2×16 kg of tetrahydrofuran. 10.53 kg of wet solid (corresponding to 8.57 kg of dry material) are obtained.

The solid obtained is ziprasidone base having a purity by HPLC of 99.2%. The global yield from the starting compound (III) is 66.3% (molar yield). Optionally, Ziprasidone base could be converted to ziprasidone hydrochloride.

### Example 4

### Preparation of Ziprasidone Base

13.26 g (0.400 mols, 3.20 molar equivalents) of sodium carbonate, 10.00 g (0.039 mols, 1.0 molar equivalent) of 3-(1-piperazinyl)-1,2-benzisothiazole hydrochloride [hydrochloride of the compound of formula (III)], 10.80 g (0.0469 mols, 1.2 molar equivalent) of 5-(2-chloroethyl)-6-chloro-1,3-dihydro-indole-2-(2H)-one [compound of formula (II) wherein X is chlorine] and 7.030 g (0.0469 mols, 1.2 molar equivalents) of NaI are added into a 250 ml round bottom, three necked reaction vessel, equipped with a reflux condenser, heating bath, anchor impeller, thermometer and under nitrogen atmosphere. At this point, 90 ml of acetonitrile are added and the mixture is heated up to reflux temperature (80° C.) for 25 hours.

The reaction is then cooled down to room temperature, stirred for 30 minutes, filtered and the cake washed with acetonitrile. A wet mixture of ziprasidone base and inorganic salts is obtained.

The resulting wet mixture is stirred with 64.6 ml of water at reflux temperature for 1 h to remove inorganic salts. The suspension is cooled down to room temperature, stirred for 30 minutes and filtered. The cake is washed with water to obtain 29.41 g of wet solid (corresponding to 14.83 g, 0.036 mol of dry material) (yield: 91.91%). At this stage, the wet solid has a chromatographic purity of 94.9% by HPLC.

The solid thus obtained is stirred with 69.8 ml of water at reflux temperature for 1 h to remove residual inorganic salts. The suspension is cooled down to room temperature, stirred for 30 minutes and filtered. The cake is washed with water to obtain 25.41 g of wet solid (corresponding to 13.48 g of crude ziprasidone base) (yield: 83.5%). At this stage, the wet solid has a chromatographic purity of 97.0% by HPLC.

### Example 5

### Ziprasidone Maleate

The free base of ziprasidone can be used in the following Examples irrespective of the process used for its production. For example, ziprasidone base produced in Examples 1-4 can be employed.

In a 1 L spherical reaction vessel, equipped with a reflux condenser, a thermometer and a magnetic stirrer, and purged with nitrogen, 400.45 ml of a tetrahydrofuran/N,N-dimethylacetamide 1:4 mixture and 60.0 g of wet ziprasidone base (corresponding to 54.8 g of dry material, 0.133 mols) are added. To the resulting suspension 24.76 g of maleic acid (1.6 molar equivalents) are added, and it is stirred for 5 minutes. At this point, 8.0 g of active charcoal are added to the deep red suspension. After stirring for 30 minutes, the suspension is filtered over celite and the solid is washed twice with 40 mL of the same solvent mixture. A clear red solution of Ziprasidone maleate is obtained, which can subsequently be converted to its hydrochloride salt by conventional means.

### Example 6

### Ziprasidone Acetate

In a 100 ml spherical reaction vessel, equipped with a thermometer and a magnetic stirrer, and purged with nitrogen, 5.96 g of wet ziprasidone base (corresponding to 4 g of dry ziprazidone base) and 16 ml of acetic acid are added. After fifteen minutes of stirring a solution is obtained. At this point, 0.04 g of active charcoal is added. After stirring for 30 minutes, the suspension is filtered over celite and the solid is washed twice with 2 mL of acetic acid. A clear brown solution of Ziprasidone acetate is obtained, which can subsequently be converted to its hydrochloride salt by conventional means.

### Example 7

### Preparation of Anhydrous Ziprasidone Hydrochloride

In a 100 ml spherical reaction vessel, equipped with a thermometer and a magnetic stirrer, and purged with nitrogen, the solution of Ziprasidone acetate obtained in example 6 is charged. After adding 0.99 ml of 36.18% aqueous hydrochloric acid (1,2 molar equivalents) to the solution, a pink suspension is obtained. It is stirred for two hours, filtered and the solid is washed twice with 2 ml of acetic acid. The solid is dried in vacuum at 40° C. until constant weight to obtain 3.49 g of anhydrous Ziprasidone hydrochloride. Global yield from ziprasidone base: 80.2%.

### Example 8

### Preparation of Anhydrous Ziprasidone Hydrochloride_(Large Scale)

In a reactor vessel equipped with a mechanical stirrer 0.604 kg of tetrahydrofurane wet ziprasidone base obtained in Example 3 prior to conversion into the hydrochloride derivative (0.5 kg dry), 1.2 kg (1.3 l) of tetrahydrofuran and 4.88 kg (5.2 l) of N,N-dimethylacetamide are added. The resulting beige suspension is stirred for ten minutes and then 0.17 kg of maleic acid is added. The suspension becomes almost instantaneously an almost clear red solution. It is filtered to remove insoluble particles and the clear solution is transferred to a clean vessel, to which 323 ml of a 4.5 M solution of hydrogen chloride in isopropanol are added at a rate of 1 ml/min. The mixture is stirred for 3 hours at 20-25° C., filtered and washed twice with 1 litre of tetrahydrofuran, to obtain 0.537 kg of wet anhydrous Ziprasidone hydrochloride that corresponds to 0.521 kg of dry anhydrous Ziprasidone hydrochloride. Molar yield: 96%. Purity by HPLC: 99.9%.

1. A process for the preparation of 5-(2-(4-(1,2-benzisothiazol-3-yl)-1-piperazinyl)ethyl)-6-chloro-1,3-dihydro-2H-indol-2-one of the formula I 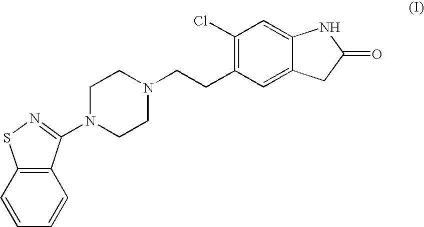
or a pharmaceutically acceptable acid addition salt, solvate, hydrate or clathrate thereof, said process comprising reacting a compound of formula II 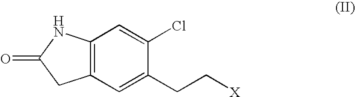
wherein X is a halogen atom, with a compound of formula III 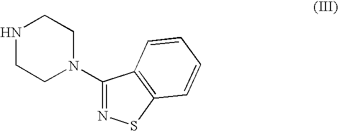
said compound of formula III being the free base or an addition salt with an organic or inorganic acid, wherein said process is characterized in that said compounds according to formulas II and III are reacted in the presence of a neutralizing agent, and are reacted in a solvent comprising acetonitrile.

2. The process according to claim 1 wherein the solvent is acetonitrile.

3. The process according to claims 1 or 2, further comprising transforming the obtained compound of formula I in a pharmaceutically acceptable acid addition salt, solvate, hydrate or clathrate.

4. The process according to any one of claims 1 to 3, wherein X is chlorine.

5. The process according to any one of claims 1 to 4, wherein compound III is an addition salt with an acid selected from hydrochloric acid, hydrofluoric acid, hydrobromic acid, hydroiodic acid, methanesulfonic acid, trifluoromethanesulfonic acid and trifluoroacetic acid.

6. The process according to claim 5, wherein the acid is hydrochloric acid.

7. The process according to any one of claims 1 to 6, wherein the neutralizing agent is selected from alkali or alkaline earth metal carbonates, bicarbonates, and tertiary amines.

8. The process according to claim 7, wherein the neutralizing agent is sodium carbonate or N,N-diisopropylethylamine.

9. The process according to any one of claims 1 to 8, wherein the compound according to formula III is used as the free base, and wherein the neutralizing agent is used in an amount of one to four molar equivalents based on the compound according to formula III.

10. The process according to any one of claims 1 to 8, wherein the compound according to formula III is used as an addition salt with an organic or inorganic acid, and wherein the neutralizing agent is used in an amount of two to four molar equivalents based on the compound according to formula III.

11. The process according to any one of claims 1 to 10, wherein the reaction is carried out in the presence of sodium iodide as a catalyst.

12. The process according to claim 11, wherein the piperazine derivative according to formula III, the alkyl halide according to formula II, the neutralizing agent and NaI are mixed in a solvent comprising acetonitrile.

13. The process according to any one of claims 11 or 12, wherein the sodium iodide is used in a closed to stoichiometric amount at or close to reflux temperature of the acetonitrile and at or close to atmospheric pressure.

14. The process according to any one of claims 1 to 12, wherein the temperature of the reaction is selected from 80° C. to 180° C.

15. The process according to claim 14, wherein the temperature of the reaction is selected from preferably 120° C. to 170° C.

16. The process according to claims 14 or 15, wherein the reaction is kept at the selected temperature for 3 to 80 h.

17. The process according to claim 16, wherein the reaction is kept at the selected temperature for 5 to 30 h.

18. The process according to any one of claims 14 to 17, wherein the process is performed in a sealed reactor.

19. The process according to any one of claims 13 to 18, wherein subsequently to keeping the reaction at the selected temperature, the reaction product is filtered and washed with acetonitrile.

20. The process according to any one of claims 1 to 19, further comprising treating the free base according to formula I with water at reflux temperature.

21. The process according to any one of claims 1 to 20, further comprising treating the free base according to formula I with tetrahydrofuran at reflux temperature.

22. Use of acetonitrile as a solvent in a process according to any one of claims 1 to 21.
